(19) United States
(12) Plant Patent Application Publication
(10) Pub. No.: US 2016/0113168 P1
(43) Pub. Date: Apr. 21, 2016

(54) Blueberry plant 'EB 9-12'

(50) ;

(71) Applicant: Vincent David Mazzardis, Joondalup (AU)

(72) Inventor: Vincent David Mazzardis, Joondalup (AU)

(73) Assignees: Pruns Persica Pty Ltd., Osborne Park (AU); Rolfe Nominees Pty Ltd., Toowoomba (AU)

(21) Appl. No.: 14/756,636

(22) Filed: Sep. 28, 2015

(30) Foreign Application Priority Data
Oct. 16, 2014 (AU) 2014/245

(51) Int. Cl. A01H 5/00

(52) U.S. Cl. PLT/157

(57) ABSTRACT

A new and distinct variety of blueberry plant, which is denominated varietally as ‘EB 9-12’ is described, and which produces large to very large sized fruit, and which further has a very good fruit flavor, and an earlier flowering, and fruit production date when grown under the ecological conditions prevailing in Yanchep Springs, Western Australia.

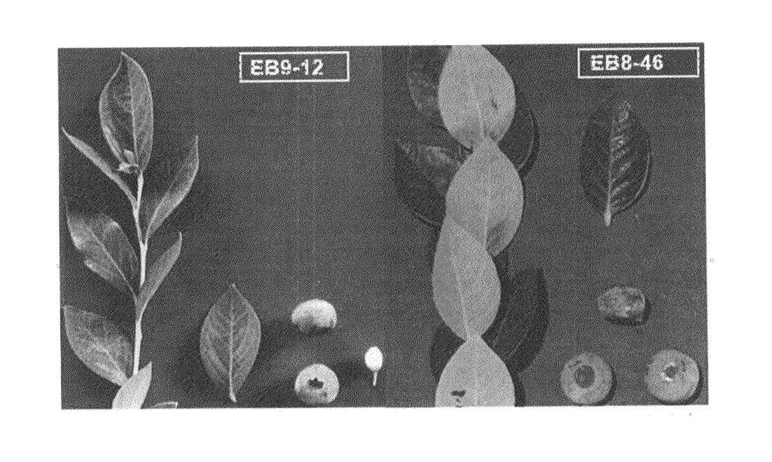

### LATIN NAME

‘Vaccinium Hybrid’

### VARIETAL DENOMINATION

‘EB 9-12’

### RELATED APPLICATION DATA

The present application claims priority to Australian Plant Breeders Rights Application, Serial No. 2014/245, and which was filed on Oct. 16, 2014, and accepted on Dec. 23, 2014.

### BACKGROUND OF THE NEW VARIETY

The present invention relates to a new, novel, and distinct variety of blueberry plant ‘Vaccinium Hybrid’ and which has been denominated varietally as ‘EB 9-12’.

### ORIGIN AND ASEXUAL REPRODUCTION OF A NEW VARIETY

The present variety of blueberry plant resulted from an ongoing development program of plant breeding. The purpose of this program is to improve the commercial quality of various plant varieties by creating and releasing promising selections of plants including blueberries. To this end, I have made both controlled and hybrid cross-pollinations each year in order to produce resulting plant populations from which improved progenies are evaluated and selected.

The blueberry plant ‘EB 9-12’ was derived from a cross-pollination employing the blueberry plant ‘BB-2’ [unpatented], and the pollen parent ‘03-6’ [unpatented] during the 2006 growing season. This first controlled cross pollination took place at Yanchep Springs, Yanchep, Western Australia. The seed parent ‘BB-2’ is characterized by an upright bush- type growth habit; a mid- to late season flowering date; and which further produces large, firm fruit. On the other hand, the pollen parent ‘03-6’ [unpatented] is characterized by a spreading growth habit, an earlier flowering date, and which further produces fruit having a large size. Seed from the seed parent ‘BB-2’ produced approximately 500 plants. These plants were then grown, and the first fruit was evaluated during the 2008 growing season. Further, an additional assessment of these same plants took place in 2009. At that time the new variety ‘EB 9-12’ was then selected for further asexual reproduction, and evaluation. The present variety was asexually reproduced by cuttings, and the plants produced from this first asexual propagation were later evaluated during the 2010 through 2014 growing seasons. The subsequent evaluations of these asexually reproduced plants show that they were true to the original plant, and it was my conclusion following those evaluations that ‘EB 9-12’ was a new, novel and distinct variety of blueberry plant.

The new, present variety of blueberry plant is characterized as to novelty by displaying a semi-upright bush-type growth habit; and which further produces large to very large and slightly flat fruit which have an excellent flavor, a very early flowering date, and an early fruit maturity date. The most closely known plant varieties which relate to the present new plant concerns the blueberry plant ‘Ridley 1111’ [unpatented]; and blueberry plant ‘EB 8-46’ (patent pending), U.S. Plant patent application Ser. No. 13/998,668, and which was filed on Nov. 21, 2013). With regard to the ‘Ridley 1111’ blueberry plant, the new variety of blueberry plant produces fruit which are considered large to very large in size, in relative comparison to the fruit produced by the ‘Ridley 1111’ blueberry plant, and which produces only medium to large fruit. On the other hand, and with regard to the blueberry plant ‘EB 8-46’, the new, present variety of blueberry plant is distinguishable therefrom by displaying a strong, to very strong plant vigor; an upright to semi-upright growth habit; a lanceolate leaf shape; a medium green, unripe fruit color; and a fruit weight which is less than that expressed by the variety ‘EB 8-46’. These distinguishing features of the closest known variety ‘EB 8-46’ as well as the distinctions relative to the parent plants ‘BB-2’ [unpatented]; and ‘03-6’ [unpatented] will be discussed in the paragraphs which follow.

### BRIEF DESCRIPTION OF THE DRAWINGS

The accompanying drawing is a color photograph of the new blueberry plant in juxtaposition relative to an image of the closest known variety ‘EB 8-46’. The photograph depicts several mature fruit of the new variety which is sufficiently matured for harvesting and shipment. The photograph also depicts a typical vegetative stem bearing typical leaves and at least one leaf showing the ventral coloration thereof, as well as two mature fruit of the present new variety. Similar plant features are seen in the image which relates to the closest known variety ‘EB 8-46’. The colors in this photograph are as nearly true as is reasonably possible on a color representation of this type. Due to chemical development, processing and printing, the leaves and fruit depicted in these photographs may or may not be accurate when compared to the actual specimen. For this reason, future color references should be made to the descriptions as provided hereinafter.

### NOT A COMMERCIAL WARRANTY

The following detailed description has been prepared to solely comply with the provisions of 35 U.S.C. §112, and does not constitute a commercial warranty, (either expressed or implied), that the present variety will, in the future, display the botanical, horticultural, or other characteristics as set forth, hereinafter. Therefore, this disclosure may not be relied upon to support any future legal claims including, but not limited to, breach of warranty of merchantability, or fitness for any particular purpose, or non-infringement, which is directed, in whole, or in part, to the present variety.

### DETAILED DESCRIPTION

Referring more specifically to the botanical details of this new and distinct variety of blueberry plant, the following has been observed during the sixth fruiting season under the ecological conditions prevailing at the farm of the inventor, and which is located near Yanchep Springs, Western Australia.
Plant:
   Plant vigor.—The present variety of blueberry plant is considered to have a strong to very strong plant vigor. This is in contrast to the closest known variety ‘EB 8-46’ and where the plant vigor characteristic which is displayed is only considered average to strong.
   Plant growth habit.—Considered upright to semi-upright. This is in contrast to the closest known variety ‘EB 8-46’ and where the plant growth habit is only considered average. In contrast to the seed parent ‘BB 2’ [unpatented], the plant growth habit of this parent plant is upright and bush-type. On the other hand, the pollen parent ‘03-6’ displays a spreading growth habit.
   One year old shoots.—Color — Green.
   Internode length.—Considered long for the species. This is in contrast to the closest known variety ‘EB 8-46’, and where this same growth characteristic is only considered average.
   Leaf length.—Considered very long for the species, about 65.80 mm., on average. This is in contrast to the closest known variety ‘EB 8-46’ where the average leaf length is about 60.33 mm.
   Leaf width.—Considered narrow for the species, about 29.93 mm. This is in contrast to the closest known variety ‘EB 8-46’, and where the average leaf width is about 34.1 mm.
   Leaf ratio.—Length/Width — Considered large to very large for the species. This is in contrast to the same growth characteristic as expressed in the closest known variety ‘EB 8-46’ and which is considered merely average.
   Leaf shape.—Generally — Lanceolete. This is in contrast to the closest known variety ‘EB 8-46’, and where the leaf shape is considered ovate.
   Leave color.—Dorsal Surface — Green.
   Color intensity.—Dorsal Surface — In relative comparison to the leaf color as expressed in the closest known variety ‘EB 8-46’, the dorsal leaf color of the present variety is only considered average, as compared to the medium to dark green color as seen in the leaves produced by the blueberry plant ‘EB 8-46’.
   Leaf marginal edge.—Generally — Considered entire.
Flowers:
   Flower bud coloration.—The anthocyanin coloration is considered to be very weak.
   Inflorescence length.—Considered average for the species.
   Corolla shape.—Urceolate.
   Corolla tube.—Size — Considered average for the species.
   Corolla tube coloration.—Generally speaking any anthocyanin coloration of the corolla tube is considered absent or very weak.
   Corolla tube ridges.—Present.
Fruit:
   Fruit cluster density.—Generally considered medium to dense for the species.
   Unripe fruit color intensity.—Considered a very light green. This is in contrast to the coloration as expressed on the unripe fruit of the closest known variety “EB 8-46’, and where this coloration is considered an average green.
   Fruit size.—Considered large to very large for the species. In this regard, the average fruit diameter is about 17.92 mm.; and the average fruit height is about 13.92 mm. This is in contrast to the closest known variety ‘EB 8-46’, and where the average fruit diameter is about 19.57 mm.; and the average fruit height is about 14.34 mm.
   Fruit shape when viewed in a longitudinal section.—Considered oblate.
   Sepal position/attitude.—The new variety is considered to be erect to semi-erect.
   Sepal type.—Generally — Incurving.
   Calyx basin diameter.—Considered small to medium. In this regard, the Calyx basin diameter (width) is about 7.40 mm.; and the Calyx basin depth is about 2.08 mm. This is in contrast to the closest known variety ‘EB 8-46’, and where the Calyx basin width is about 8.22 mm., and the Calyx basin depth is about 3.38 mm.
   Fruit.—Intensity of Fruit Bloom — Considered strong to very strong.
   Fruit skin color.—Considered dark blue.
   Fruit.—Firmness — Considered firm to very firm.
   Fruit weight.—Generally speaking about 2.85 gm., on average. This is in contrast to the average weight of the fruit produced by the closest known variety ‘EB 8-46’, and which weighs about 3.76 gm.
   Fruiting type.—Generally speaking fruiting occurs on one-year old and current season's shoots.
   Vegetative bud burst.—Generally speaking, the time of vegetative bud burst is considered early to average in the season as compared to other varieties. The beginning of flowering on one-year old shoots, generally speaking occurs very early to early in the season. This is in contrast to the seed parent ‘BB 2’, and where this plant characteristic occurs in the middle to late in the season; and with respect to the pollen parent ‘03-6’, and where this plant same plant characteristic is manifested early in the season.
   Beginning of flowering on current year's shoots.—This is considered to occur early in the season.
   Beginning of fruit ripening on one-year old shoots.—Generally speaking this is considered to occur early to sometimes as late as the middle of the season.
   Beginning of fruit ripening on current year's shoots.—This is considered to occur early in the season.
   Fruit sweetness.—Considered high to very high for the species.
   Resistance to insects and diseases.—No particular susceptibilities were noted. The present blueberry variety has not been tested to expose or detect any susceptibilities or resistances to any known plant and/or fruit diseases. Although the new variety of blueberry plant possesses the described characteristics when grown under the ecological conditions prevailing near Yanchep Springs, Western Australia, it should be understood that the variations of the usual magnitude and characteristics incident to changes in growing conditions, fertilization, pruning, pest control, frost, climatic variables and horticultural management are to be expected.

Having thus described and illustrated my new variety of blueberry plant, what I claim is new, and desire to secure by plant Letters Patent is:

1. A new and distinct variety of blueberry plant substantially as illustrated and described, and which is characterized principally as to novelty by producing a large to very large fruit which has a good flavor, and an earlier flowering and fruit production dates, when grown under the ecological conditions prevailing near Yanchep Springs, Western Australia.
